(19) United States
(12) Patent Application Publication
(10) Pub. No.: US 2002/0031829 A1
(43) Pub. Date: Mar. 14, 2002

(54) Arrayed collection of genomic clones

(76) Inventors: Brian Zambrowicz, The Woodlands, TX (US); Arthur T. Sands, The Woodlands, TX (US)

Correspondence Address: LEXICON GENETICS INCORPORATED, 4000 RESEARCH FOREST DRIVE, THE WOODLANDS, TX, 77381, US

(21) Appl. No.: 09/930,877

(22) Filed: Aug. 14, 2001

Related U.S. Application Data
(60) Provisional application No. 60/225,244, filed on Aug. 15, 2000.

(51) Int. Cl. C12N 15/87; C07H 21/04; C12N 1/21; C12N 1/18; A01K 67/00

(52) U.S. Cl. 435/455; 435/254.1; 435/252.3; 435/320.1; 800/21; 536/23.1

(57) ABSTRACT

Novel collections of isolated genomic clones are described that are incorporated into gene targeting cloning vectors. The described collections find particular application in gene discovery, the production of mutated cells and animals, and gene activation.

The present application claims the benefit of U.S. Provisional Application No. 60/225,244 which was filed on Aug. 15, 2000 and is herein incorporated by reference in its entirety.

### 1.0 FIELD OF THE INVENTION

The present invention relates to methods, vectors, and collections of recombinant constructs incorporating structural elements that substantially enhance the ease and rapidity of effecting gene targeting of a eukaryotic chromosome. Such methods are important for engineering specific gene mutations, construction of conditional knockouts, inducible gene expression or regulation, shuttling nucleic acid sequences throughout the genome, and gene activation or over expression.

### 2.0. BACKGROUND OF THE INVENTION

The pending release of the first mammalian genome to be comprehensively sequenced and assembled marks an important milestone in the modern era of genetic research. However, the annotated human genomic sequence evinces a startling absence of bona fide functional information describing the roles of the various genes (or often predicted genes) in mammalian physiology. Such physiological information is of critical importance because opportunities for medical intervention typically involve therapeutic interventions that alter or other wise regulate mammalian physiology. Given that ethical and practical concerns proscribe genetic experimentation in humans, scientists have often had to resort to the study of cell lines in culture and to then extrapolate the information derived from the study of individual cells into theoretical predictions about what the cell-based data might mean within the far more complex context of mammalian biology.

The inherent limitations of such cell based approaches have led other scientists to branch out into higher throughput, but less meaningful, means of studying gene function (i.e., chips, yeast, etc.). Alternatively, some scientists have used lower throughput, but more informative classical molecular genetic models (i.e., flies, worms, fish, etc.) to glean information about gene function in the context of living, albeit primitive, multicellular organisms. Although classical genetic models generally provided information of limited value, the fact that they allowed for proactive genetic intervention and study was apparently deemed superior to the alternative approach of passively gathering and sorting statistics about human physiology from the patient population, and then spending years searching for the human gene or genes that may be involved.

Over ten years, and in some cases many decades, of scientific experience using the approaches described above has demonstrated the inherent limitations of using the above methods to broadly study human gene function. Consequently, mammalian model systems that allow for the direct intervention and study of mammalian physiology (e.g., cardiopulmonary system, nephrology, immune function, bone and muscle function, thermoregulation, behavior, etc.) have emerged as the animal models of choice for studying human gene function. Of these mammalian model organisms, a particular animal of choice is the mouse.

### 3.0. SUMMARY OF THE INVENTION

Most genomic libraries used in molecular biology are generated and stored as a milieu of pooled clones that are subsequently screened by high density methods such as plaque lifts and colony hybridization. Although effective, such traditional methods are less well suited for high-throughput commercial applications where substantial production efficiencies are highly desirable, and can be used to amortize substantial up front costs associated with a given method of production.

The present invention relates to the construction of a commercial-scale collection of isolated mammalian genomic clones that are individually arrayed and stored in solid support matrices such as, for example, the wells of micro titer plates, and methods of using of such clones to construct gene targeting constructs suitable for genetically engineering the chromosome of target cells by targeted homologous recombination. In a particularly preferred embodiment, such methods include the use of the isolated genomic clones in gene targeting where at least one selectable marker that can be negatively selected in the target cell is present such that it flanks, or other wise defines, one or more ends of the genomic insert used to construct the targeting vector. In a yet more preferred embodiment, the negative selectable marker(s) can be present on the vector such that the genomic inserts present in the collection of individually isolated mammalian genomic clones are flanked on one or both ends by one or more negatively selectable marker(s).

Preferably, the collection of individually isolated genomic clones comprises a sufficient number of clones to provide at least about two fold redundancy, preferably at least about five fold, and more preferably at least about nine-to-ten fold redundancy or more to help ensure that a representative clone is present in the library for most, if not all, regions of the mammalian genome used to generate the genomic library.

In a particularly preferred embodiment, the genomic insert within the clones present in the collection is at least partially sequenced such that a minimum of about 100 bases of DNA sequence has been obtained which can be used to “tag” and track the clone of interest. A collection of such sequence tags can then be used as an sequence-based index for the collection of clones.

Another embodiment of the present invention relates to the use of the described collection of clones to effect the gene targeted genetic engineering of embryonic stem cells and the use of such cells to produce genetically engineered animals.

Yet another embodiment of the present invention relates to the use of the described collection of mammalian clones to effect the targeted activation of gene expression in mammalian, including human, cells in culture, and the use of such cells, or the genetic materials from such cells, to produce therapeutic products.

### 4.0. DETAILED DESCRIPTION OF THE INVENTION

The present invention relates to an arrayed collection of individually isolated genomic clones that have been rationally designed and arrayed to allow for the rapid screening and identification of the clone of interest by, for example, polymerase chain reaction (PCR).

The described isolated clones can also be directly indexed by sequence tagging. Where sequence tagging is desired, one or more unique priming sequences are present on one or both regions of the vector that flank that genomic insert to allow for the specific binding of synthetic oligonucleotides that are used to prime sequencing reactions. Once sequence tagged, the individually isolated and stored clones can be tracked, analyzed, and searched “in silico” using a computer database and associated bioinformatics tools. Such sequence tags are particularly useful when one desires to rapidly obtain a targeting vector corresponding to a region described in the sequence data from the human and mouse genome sequencing efforts (the tag allows for the clone of interest to be directly identified). Alternatively, the sequence information in the tag can be correlated with genomic sequence data and “microchip” expression data to identify and prioritize alleles for further development and study by gene targeting (i.e., the production of knockout animals or other genetically engineered animals).

By individually isolating, arraying, and preferably sequencing, the genomic clones present in the collection, a commercial scale functional genomic resource results that substantially streamlines the efforts required to construct the complex gene targeting vectors that are required for, inter alia, the production of conditional mutations, precise frame shift or nonsense mutations, point mutations, deletion mutations, gene replacement projects, and targeted gene activation. Consequently, the present invention complements commercial scale functional genomics technologies such as those described in U.S. Pat. No. 6,080,576, and U.S. application Ser. No. 08/942,806 both of which are herein incorporated by reference in their entirety.

The arraying of individually isolated genomic clones can also provide an alternative to sequence tagging. Multiple plates can be combined into one or more arrays (e.g., columns and rows) and individual clones are pooled by row and by column. For example, 96 well plates of individual clones may be arranged adjacent to each other to provide a larger (or virtual/figurative) two dimensional grid (e.g., four plates may be arranged to provide a net 16×24 grid, etc.), and the various rows and columns of the larger grid may be pooled to achieve substantially the same result. Similarly, plates can simply be stacked, literally or figuratively, or arranged into a larger grid and stacked to provide three dimensional arrays of individual clones. Representative pools from all three planes of the three dimensional grid may then be analyzed, and the three positive pools/planes can be aligned to identify the desired clone. For example, ten 96 well plates may be screened by pooling the respective rows and columns from each plate (a total of 20 pools) as well as pooling all of the clones on each specific plate (10 additional pools). Using this method, one can specifically identify a desired clone from a pool of, for example, 960 clones by performing PCR (using primers designed from genomic sequence) on only 30 pooled samples. Of course, the above arraying examples can be combined (up to the practical limits of detection) to, for example, theoretically allow for the identification of a specific clone from 201,600 samples in several hours using only 176 PCR reactions (assuming pooling of rows, columns, from a 7-high×5-long virtual 2-D array of 96 well plates that has been virtually stacked and pooled in each stacked plane 60 high). Total clone pools from twenty of such arrays could be preliminarily screened by PCR to allow the two step identification of a specific clone from a collection of over 4 million individual clones using as few as 196 PCR reactions (20 PCR reactions to identify a positive pool/array followed by 176 reactions to identify the specific clone of interest). A similar pooling/screening strategy can be employed using DNA pools that have been affixed to support membranes and screened (and stripped and rescreened) by high stringency hybridization.

In a particularly preferred embodiment, the isolated clones in the collection are present within a vector that has been engineered to flank the genomic insert with one or more markers on one or both ends that can be used to negatively select for or against, or otherwise used to identify, mammalian cells incorporating and expressing such markers. In the case of negatively selectable markers, cells expressing such markers are either killed, or are identified by the presence of the marker and, given that the presence of the negative marker indicates that the desired targeting event has not occurred, not selected for further use/analysis. Specific examples of markers that can be used to identify and/or negatively select cells harboring such markers include, but are not limited to, the thymidine kinase (TK) gene, ricin toxin, green fluorescent protein, luciferase, chromogenic markers, beta galactosidase, diphtheria toxin, and the hypoxanthine phosphoribosyl transferase (HPRT) as well as markers encoding similar biochemical activities and other markers such as those outlined in U.S. Pat. No. 5,487,992 herein incorporated by reference in its entirety.

The individually isolated genomic clones of the present invention can be stored using any of a wide variety of traditional means. For example, the genomic clones can be stored as phage, preferably bacteriophage lambda, cosmids, plasmids, and can be stored as constructs within living bacterial hosts (e.g., “stabs”, glycerol or DMSO stocks of E. coli, etc.), as “naked” DNA constructs, or as phage preparations.

The individually isolated genomic clones present in the described collection can be stored in individual containers or stored as arrays on, for example, 96 or 384 well microtiter plates, or similar support matrices including higher density formats (which may include biological media where live bacteria harboring the clones are to be stored). Preferably, the storage media are amenable to robot or other automated forms of manipulation and data tracking.

Generally, the number of clones present in the collection shall be a function of the extent to which one desires to represent, or over-represent, the mammalian genome of interest, and the average size of the genomic DNA inserts present in the vectors used to construct the collection. Preferably, the size of the genomic inserts shall be, on average, between about 1 kb and about 35 kb in length, more preferably between about 3 kb and about 20 kb in length, more preferably about 5 and about 15 kb, and more preferably still between about 8 kb and about 12 kb. Assuming an average genomic insert size of approximately 10 kb, and assuming that there are approximately 3×10⁹ bases in an average mammalian genome, approximately 300,000 random clones would be necessary to represent a single pass representation of the genome. Consequently, approximately 3,000,000 individual clones would be necessary to represent a 10 fold over representation of the mammalian genome. Such numbers are readily manageable as shown by, for example, the well publicized methods and efforts relating to the human genome project and competing private commercial enterprises. The presently described collection, methods, and vectors are ideally suited to the implementation of commercial scale sequencing efforts, and effectively represent a functional genomics resource that is well suited to be developed and used in conjunction with such efforts.

Although mammalian genomic libraries have been specifically described (e.g., pigs, goats, cows, rodents, humans, sheep, etc.), the present invention is equally applicable to virtually any eukaryotic cell that can be manipulated by gene targeting. For example, collections of the described individually isolated genomic clones, preferably flanked by suitable negative selectable markers, can be used to construct indexed arrays of gene targeting vectors in primary animal tissues, including birds and fish, as well as any other eukaryotic cell or organism including, but not limited to, yeast, insects, worms, molds, fungi, and plants. Plants of particular interest include dicots and monocots, angiosperms (poppies, roses, camellias, etc.), gymnosperms (pine, etc.), sorghum, grasses, as well as plants of agricultural significance such as, but not limited to, grains (rice, wheat, corn, millet, oats, etc.), nuts, lentils, tubers (potatoes, yams, taro, etc.), herbs, cotton, hemp, coffee, cocoa, tobacco, rye, beets, alfalfa, buckwheat, hay, soy beans, sugar cane, fruits (citrus and otherwise), grapes, vegetables, and fungi (mushrooms, truffles, etc.), palm, maple, redwood, yew, oak, and other deciduous and evergreen trees.

After identification, in order to effect gene targeting the described clones are typically modified to insert at least one genetic marker that allows for the positive selection of gene targeted cells that incorporate and express the marker. Examples of such markers include, but are not limited to, neo, puro, his, beta galactosidase, green fluorescent protein, luciferase, as well as other markers described in, for example, U.S. Pat. No. 5,487,992, as well as markers known in the art may be described in Sambrook et al. (1989) Molecular Cloning Vols. I-III, Cold Spring Harbor Laboratory Press, Cold Spring Harbor, N.Y., and Current Protocols in Molecular Biology (1989) John Wiley & Sons, all Vols. and periodic updates thereof, herein incorporated by reference). The described positive selection markers can be introduced into the genomic inserts using molecular biology techniques or by exploiting the homologous recombination machinery of living cells such as bacteria and yeast. The use of yeast homologous recombination is described in U.S. application Ser. No. 09/171,642 filed Sep. 21, 1998 and Storck et al., 1996, Nucleic Acids Res., 24(22):4594-4596 which are both herein incorporated by reference in their entirety. Additional methodologies that can be employed to construct gene targeting vectors using the described collection include, but are not limited to, systems employing transposon mediated gene targeting as described in U.S. application Ser. No. 60/049,523, filed Jun. 13, 1997 herein incorporated by reference in its entirety, and systems using bacterial recombination as described in Angrand et al., 1999, Nucleic Acids Res. 27(17):e16 herein incorporated by reference in its entirety.

Typically, the presently described targeting constructs (usually after suitable engineering to insert a positive selectable marker) can be introduced to target cells by any of a wide variety of methods known in the art. Examples of such methods include, but are not limited to, electroporation, viral infection, retrotransposition, microinjection, lipofection, transfection, or as non-packaged/complexed or “naked” DNA.

When such cells are totipotent embryonic stem cells, the engineered cells can be microinjected into blastocysts and implanted in suitable pseudopregnant host animals to produce chimeric offspring that can be used to subsequently breed and produce offspring capable of germ line transmission of the genetically engineered allele (see generally, U.S. Pat. No. 6,087,555 herein incorporated by reference in its entirety).

In addition to the production of gene targeted animals, the described collections of isolated genomic clones can be to used to allow for the rapid construction of targeted human gene activation cassettes as well as vectors for gene therapy. Preferably, the targeting regions of the described genomic clones are isogenic with the targeted region of the chromosome of the targeted cells or tissues (see U.S. Pat. No. 5,789,215 herein incorporated by reference in its entirety).

The present invention is further illustrated by the following examples, which are not intended to be limiting in any way whatsoever.

### 5.0. EXAMPLES

### 5.1. Construction of the Collection of Clones

Murine genomic DNA was cleaved by partial digestion with Sau3A and fragments of between about 10-15 kb were isolated and cloned into a linearized lambda KOS vector. Alternatively, the genomic fragments could be generated by mechanically shearing the DNA. The resulting phage clones are then used to infect bacteria expressing Cre-recombinase to produce a library of clones present in a circular E. coli/yeast shuttle vector (pKOS). The colonies of bacteria harboring the plasmid clones are subsequently picked and replicated onto microtiter plates for storage, and further processing and analysis. Plasmids are then isolated from the bacterial clones and are then distributed onto additional plates for storage, generation of appropriate pools, and/or analysis (sequencing, etc.). Any resulting DNA sequences are then stored in a relational database and used as an storage index that can be used to track and retrieve specific clones.

### 5.2. Construction of Mutated Cells and Animals from Clones

When the collection of individually isolated genomic clones has been tagged by DNA sequencing, DNA sequence data can be used to electronically screen and identify the clone(s) of interests in the library. Alternatively, oligonucleotides generated from a query sequence can be used to prime PCR reactions for screening for and identifying specific clones of interest from the arrayed pools.

Once identified, the specific genomic clone of interest can be expanded, and used to construct a gene targeting vector suitable for positive/negative selection essentially as described in U.S. application Ser. No. 09/171,642. Where ES cells have been targeted, the cells can be used to generate genetically engineered animals that are heterozygous and/or homozygous for the targeted allele and capable of germline transmission of the targeted allele.

All publications and patents mentioned in the above specification are herein incorporated by reference. Various modifications and variations of the described invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of animal genetics and molecular biology or related fields are intended to be within the scope of the following claims.

What is claimed is:

1. A collection of genomic DNA clones that have been individually isolated and arrayed unto a solid support matrix wherein each of said clones is present in a vector comprising a marker sequence encoding an activity negatively selectable in mammalian embryonic stem cells.

2. A collection of genomic DNA clones according to claim 1 wherein the genomic component of said clones has been sequenced for at least about 75 bases in from one or both ends of the genomic sequence present in the vector, and wherein said vector encodes a marker sequence encoding an activity negatively selectable in mammalian embryonic stem cells.

3. A collection according to claim 2 comprising at least about 500 clones.

4. A collection of genomic DNA clones that have been individually isolated and arrayed unto a solid support matrix wherein each of said clones is represented in at least three distinct pools of clones that can be screened to precisely locate a clone of interest present in the collection.

5. A process of generating a gene targeted animal or cell using a clone obtained from a collection according to any on of claims 1, 2, 3, or 4.

6. A process according to claim 5 wherein said clone is modified by homologous recombination in yeast or bacteria.

7. A process according to claim 5 wherein said clone is modified by transposition.
